(19) United States
(12) Patent Application Publication
(10) Pub. No.: US 2021/0393361 A1
(43) Pub. Date: Dec. 23, 2021

(54) DEVICE FOR ROBOT-ASSISTED SURGERY

(71) Applicant: avateramedical GmbH, Jena (DE)

(72) Inventors: Tobias Keim, Berlin (DE); Michael Jahn, Berlin (DE); Tobias Django Baudisch, Berlin (DE)

(73) Assignee: avateramedical GmbH, Jena (DE)

(21) Appl. No.: 17/350,578

(22) Filed: Jun. 17, 2021

(30) Foreign Application Priority Data
Jun. 19, 2020 (DE) 1020201162561

(51) Int. Cl. A61B 50/00; A61B 34/30; A61B 34/00; B25J 19/00; B25J 13/08

(52) U.S. Cl. CPC A61B 50/00; A61B 34/30; A61B 2034/301; B25J 19/0045; B25J 13/088; A61B 34/73

(57) ABSTRACT

A device for robot-assisted surgery comprises at least one manipulator arm with a non-sterile coupling unit comprising at least one first drive element. Further, the device comprises a sterile instrument unit arranged in a sterile area and comprising at least one second drive element arranged rotatably around an axis of rotation. The first drive element and the second drive element are configured and arranged in a coupled state such that by the first drive element a force can be exerted on the second drive element, for rotation of the second drive element about the axis of rotation, and in the coupled state the first drive element and the second drive element are arranged side by side in a plane of rotation orthogonally to the axis of rotation. Further, the device comprises a sterile barrier which is arranged at least between the first drive element and the second drive element.

### CROSS-REFERENCE TO RELATED APPLICATIONS

This application claims the benefit of German Application 10 2020 116 256.1, filed on Jun. 19, 2020, both of which are incorporated herein in their entireties.

### TECHNICAL FIELD

The invention relates to a device for robot-assisted surgery comprising at least one manipulator arm with a non-sterile coupling unit having at least one first drive element. The device further has at least one sterile instrument unit arranged in a sterile area and comprising at least one second drive element arranged to be rotatable about an axis of rotation. The instrument unit is couplable to the coupling unit.

### BRIEF DESCRIPTION

In minimally invasive surgery, so-called telemanipulator systems, also referred to as robot assistance systems, are increasingly used for robot-assisted surgery. The sterile surgical field is protected from the non-sterile elements of the telemanipulator system with the aid of a sterile cover. The sterile cover prevents contamination of the sterile surgical field as well as contamination of the telemanipulator system by body fluids and/or tissue of the operated patient or the surgical personnel. This reduces the risk of cross-contamination.

With the aid of the telemanipulator system, surgical instruments and/or endoscopes are controlled in their position and orientation on the basis of operator inputs and inevitably come into physical contact with the patient to be operated so that the surgical instruments and/or endoscopes become contaminated with body fluids and/or tissue of the patient to be operated. At the same, the surgical instruments must be mechanically, electrically and/or optically coupled to the telemanipulator system in order to be able to realize active positioning and alignment of the surgical instrument as well as a desired actuation of a surgical instrument. For this purpose, a coupling interface, which can be designed as a coupling unit, is provided on each manipulator arm.

The material used during a surgical procedure, including the surgical apparatuses and instruments used and the other components of the telemanipulator system, can be divided into three categories:

Category 1: The material is sterile and becomes contaminated during the surgical procedure. The material is disposed of after the operation. This means that the material is used only once.

Category 2: The material is sterile, is contaminated during the surgical procedure and is cleaned and sterilized after the operation. This means that the material is used more than once. Such multiple-use materials must be designed and produced in accordance with the requirements for processable sterilizability.

Category 3: The material is not sterile. During the surgical procedure, contamination of the sterile surgical field is prevented by sterile covering and wrapping. At the same time, the non-sterile material is protected from contact with body fluids and/or tissue.

If it is necessary to couple category 1 or category 2 devices with category 3 devices, a sterile interface and/or a sterile barrier is required to prevent contamination of the category 1 or category 2 devices by the non-sterile category 3 devices and, conversely, to prevent contamination of the category 3 devices, since these are generally technically designed as non-sterilizable and non-autoclavable components. The design of devices as sterilizable and autoclavable components requires a special technical design of the device for the sterilization process, so that a higher development effort as well as a considerable validation effort are required to prove the effectiveness of the sterilization process. For such proof, it is particularly necessary to contaminate and sterilize the device several times in succession, to carry out an effectiveness test of the sterilization and to carry out a functional test after sterilization has been completed. This requires proof that the devices could be safely sterilized and thus reused after each sterilization.

From document U.S. Pat. No. 7,666,191 B1, a telemanipulator system is known in which the non-sterile manipulator arms are covered with the aid of a sterile foil. The coupling unit of the manipulator arm comprises four rotary actuators that are coupled to a first side of a sterile adapter integrated in the sterile foil. With the aid of the sterile adapter, the rotary movements of the four rotary actuators of the coupling unit of the manipulator arm are coupled in engagement with four rotatably mounted transmission means integrated in the sterile adapter. On the sterile exterior of the sterile adapter, these sterile transmission means can be engaged with driven elements of the sterile surgical instrument. Furthermore, electrical signals can be transmitted between the inside and the outside of the sterile adapter via this sterile adaptor.

Thus, the sterile adapter prevents the rotary actuators and the electrical connections of the sterile surgical instrument from coming into direct contact with the rotary actuators and the electrical connections of the coupling unit of the non-sterile manipulator arm. Contamination of the surgical instrument by contact with non-sterile parts of the manipulator arm is prevented by the sterile adaptor. However, this solution requires the sterile adapter to have rotatably mounted transmission means, as well as transmission means for transmitting electrical signals, which makes the adapter costly to manufacture and prone to failure. In particular, it is costly to ensure the rotatability of the transmission means and the impermeability of the bearing of the transmission means in the sterile adaptor when the transmission means come into contact with body fluid. The sterile adaptor itself is intended for single use as part of the sterile foil.

From document EP 3025667 A1, a sterile lock is known that shields drive elements of a coupling unit from a sterile surgical area in a sterile manner before a sterile instrument unit is connected to the sterile lock and after the sterile instrument unit is disconnected from the sterile lock. Further, the sterile instrument unit has sterile flaps that shield drive elements of the instrument unit from a sterile surgical area in a sterile manner before the sterile instrument unit is connected to the sterile lock and after the sterile instrument unit is disconnected from the sterile lock.

In principle, each element in the functional chain for coupling the manipulator arm and the instrument is a potential source of error and is associated with additional costs. The more complex a sterile lock or a sterile adaptor is designed, in particular the more moving elements are provided, the more sources of error occur. Furthermore, the known sterile locks and sterile adapters have bearings and/or abutting surfaces of flaps, the impermeability of which must be ensured.

It is the object of the invention to specify a device for robot-assisted surgery in which a sterile coupling of a coupling unit of a non-sterile manipulator arm with a sterile instrument unit arranged in a sterile area is easily possible, wherein a reliable transmission of force between the coupling unit and the instrument unit is possible and a sterile barrier shields non-sterile elements from the sterile area.

This object is solved by a device having the features of claim 1. Advantageous embodiments are specified in the dependent claims.

A device for robot-assisted surgery comprises at least one manipulator arm with a non-sterile coupling unit having at least one first drive element. Further, the device comprises a sterile instrument unit arranged in a sterile area, which comprises at least one second drive element arranged rotatably about an axis of rotation, wherein the instrument unit can be coupled to the coupling unit of the manipulator arm. The first drive element and the second drive element are configured and arranged in a coupled state such that a force can be exerted by the first drive element on the second drive element to rotate the second drive element about the axis of rotation. In the coupled state, the first drive element and the second drive element are arranged next to each other in a plane of rotation perpendicular to the axis of rotation. Furthermore, the device comprises a sterile barrier arranged at least between the first drive element and the second drive element. This enables a particularly compact and simple design. In particular, the design of the sterile barrier is particularly simple. The first drive element can also be referred to as the coupling unit drive element and the second drive element as the instrument drive element.

It is advantageous if, in the coupled state, the first drive element is arranged in the plane of rotation of the second drive element such that a surface of the first drive element facing the second drive element runs parallel to a cylindrical lateral surface or a cylindrical enveloping surface of the second drive element in at least one area. This makes it possible for the first drive element to exert the force on the second drive element in a particularly safe manner.

It is advantageous if magnetic field-generating elements are arranged in the plane of rotation on a side of the first drive element facing the second drive element and magnetic field-generating or magnetic elements of the second drive element are arranged on a circular path around the axis of rotation. This enables a particularly safe drive of the second drive element.

It is particularly advantageous if the magnetic field-generating elements of the first drive element are electromagnets and the magnetic field-generating elements of the second drive element are electromagnets with circumferentially alternating poles, or if the magnetic field-generating elements of the first drive element are electromagnets and the magnetic elements of the second drive element are permanent magnets with circumferentially alternating poles. This enables a particularly compact and robust design.

It is particularly advantageous if magnetic fields are generatable in the first drive element with the aid of electromagnets, by which magnetic fields the force can be exerted on the permanent magnets of the second drive element, wherein the second drive element rotates by changing the polarity of the electromagnets and/or changing a switching state of the electromagnets. This enables a reliable drive of the second drive element.

It is advantageous if the first drive element comprises at least one movable and/or deformable actuator which is movable and/or deformable such that the actuator contacts a lateral surface of the second drive element at least temporarily such that the force can be exerted on the second drive element by frictional connection and/or positive connection between the actuator and the lateral surface of the second drive element. This results in a particularly safe transmission of force from the first to the second drive element.

It is particularly advantageous if the actuator is at least one piezoelectric actuator and with which, by way of a frictional connection and/or positive connection between the actuator and the lateral surface of the second drive element, the force can be exerted on the second drive element. As a result, a particularly powerful drive of the second drive element is achieved.

It is advantageous if the surface of the first drive element facing the second drive element runs along an arc around the axis of rotation with a center angle of 45° to 180°. This ensures a particularly robust drive of the second drive element.

It is advantageous if the sterile barrier has a continuously closed surface at least in the area between the first drive element and the second drive element. This ensures a particularly high level of safety for preventing contamination of the sterile area.

It is advantageous if a section of the sterile barrier arranged between the first drive element and the second drive element runs along an arc around the axis of rotation. This provides a particularly safe arrangement of the sterile barrier.

It is advantageous if the coupling unit comprises at least one sensor unit for detecting an angle of rotation of the second drive element. In this way, particularly precise control over the movement of the second drive element is achieved.

It is advantageous when the sensor unit comprises an optical sensor which detects a coded, optically detectable pattern on the second drive element circulating in a plane of rotation, each detectable angle of rotation of the second drive element being assigned a part of the pattern which is detectable by the sensor at this angle of rotation, each part of the pattern which can be detected by the sensor at one angle of rotation being uniquely different from other parts of the pattern which are detectable by the sensor at other angles of rotation. In this way, a particularly reliable detection of the angle of rotation is achieved.

It is advantageous if, in a coupled state, the second drive elements are arranged in a plurality of planes of rotation one above the other along the same axis of rotation, and each first drive element is arranged in a plane of rotation in pairs with a second drive element, the number of pairs of drive elements being at least two, in particular three or four. Thus, a plurality of degrees of freedom of an end effector of the instrument unit are possible.

It is particularly advantageous if a plurality of sensor units are provided, that the number of sensor units corresponds at least to the number of second drive elements, and that at least one sensor unit each detects the angle of rotation of a second drive element. In this way, a particularly precise and reliable control of the end effector is achieved.

It is advantageous when the instrument unit comprises an instrument with an end effector arranged at a distal end of an instrument shaft, wherein the at least one second drive element is coupled to the end effector and wherein the end effector is movable and/or controllable in at least one degree of freedom with the aid of the at least one second drive element, in particular, in the case of four second drive elements, in four degrees of freedom, wherein in each case two of the second drive elements effect a rotary movement about the longitudinal axis of the instrument shaft and in each case two further second drive elements effect a longitudinal movement in the direction of the longitudinal axis of the instrument shaft. Alternatively, it is advantageous if the instrument unit comprises an endoscope with an endoscope shaft, wherein the at least one second drive element is coupled to the endoscope, the endoscope shaft and/or an optical system of the endoscope such that a movement of the endoscope, the endoscope shaft and/or the optical system is possible in at least one degree of freedom with the aid of the at least one second drive element. This enables a particularly large variety of different instrument units and a particularly flexible use of the device.

Further features and advantages result from the following description, which explains embodiments in more detail in connection with the enclosed Figures.

### BRIEF DESCRIPTION OF THE DRAWINGS

FIG. 1 shows a schematic illustration of a device for robot-assisted surgery.

FIG. 2 shows a part of a manipulator arm of the device according to FIG. 1 with a coupling unit as well as a sterile barrier and an instrument unit according to a first embodiment in a non-coupled state.

FIG. 3 shows a part of the manipulator arm with the coupling unit as well as the sterile barrier and the instrument unit according to FIG. 2 in a coupled state.

FIG. 4 shows a schematic exploded view of the instrument unit.

FIG. 5 shows a side view of the instrument unit.

FIG. 6 shows an instrument unit according to a second embodiment.

FIG. 7 shows an instrument unit according to a third embodiment.

FIG. 8 shows a coupling unit and an instrument unit with an electromagnetic drive according to a fourth embodiment.

FIG. 9 shows a schematic drawing of magnetic field-generating elements of an electromagnetic drive of the coupling unit and the instrument unit according to FIG. 8 in a first state with a first polarization.

FIG. 10 shows a schematic drawing of the electromagnetic drive according to FIG. 9 in a second state with a second polarization.

FIG. 11 shows a coupling unit and an instrument unit according to a fifth embodiment with a mechanical force transmission.

FIG. 12 shows a schematic drawing of engagement elements of the coupling unit and of the instrument unit according to FIG. 11 in a first step.

FIG. 13 shows a schematic drawing of the engagement elements according to FIG. 12 in a second step.

FIG. 14 shows a coupling unit with four coupling unit drive elements and the instrument unit according to the third embodiment.

FIG. 15 shows a schematic sectional drawing of an instrument drive element according to a sixth embodiment of an instrument unit, and

FIG. 16 shows a schematic illustration of a drive of an instrument unit.

### DETAILED DESCRIPTION

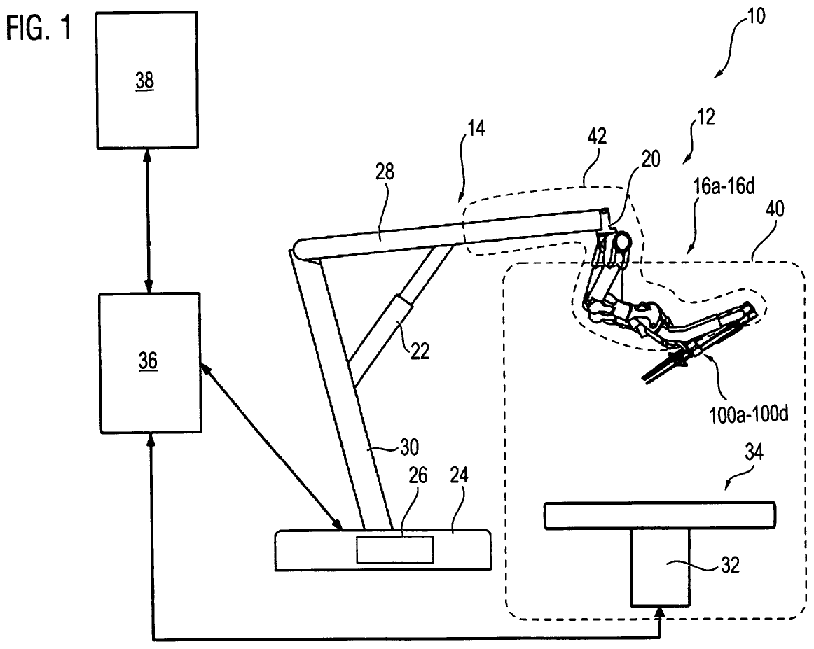

FIG. 1 shows a schematic illustration of a device 10 for robot-assisted surgery with a manipulator 12, having a stand 14 and four manipulator arms 16a to 16d. In other embodiments, the manipulator 12 may also have more or less manipulator arms 16a to 16d. Each manipulator arm 16a to 16d is connected to an instrument unit 100a to 100d via a coupling unit of the manipulator arm 16a to 16d. The instrument unit 100a to 100d is sterile and comprises a surgical instrument, in particular with an end effector, wherein the end effector may be moved and/or actuated with the aid of the coupling unit of the manipulator arm 16a to 16d. As an alternative to the surgical instrument, the instrument unit 100a to 100d may also comprise an optical instrument, in particular an endoscope, and/or a medical device, in particular for applying a drug, for dispensing an irrigation fluid and/or for aspirating an irrigation fluid and/or secretion.

The stand 14 has a stand foot 24 standing on the floor of an operating room. The manipulator arms 16a to 16d are connected to a stand head 20 of the stand 14. In other embodiments, the stand may also be a ceiling stand.

The position of the stand head 20 is adjustable with the aid of a stand arm drive unit 22 and with a stand foot drive unit 26 arranged in the stand foot 24. With the aid of the drive unit 22, the stand arms 28, 30 are movable relative to each other. With the aid of the drive unit 26, the inclination of the stand arm 30 relative to the placement surface of the stand foot 24 can be changed and/or the stand arm 30 can be rotated about a vertical axis of rotation. Generally, positioning of the stand head 20 is performed prior to surgery on a patient. During surgery, the position of the stand head 20 relative to the column 32 of an operating table 34 typically remains unchanged. The manipulator 12 is controlled with the aid of a control unit 36. The control unit 36 is connected via a data and/or control line to an input and output unit 38, which in particular outputs an image of the operation field to an operator in real time with the aid of at least one display unit. The operator makes control inputs by which the instrument units 100a to 100d are positioned and actuated during the operation of the patient. The input and output unit 38 thus serves as a human-machine interface.

The control unit 36 is further connected to a control unit of the operating table 34, which is not shown, via a control and/or data connection. This control and/or data connection ensures, among other things, that the position of the patient support surface or of segments of the patient support surface of the operating table 34 may only be changed if this is possible without danger for a patient to be operated due to the positioning of the instrument units 100a to 100d.

The operating table 34 and the instrument units 100a to 100d are arranged in a sterile operating area 40. The manipulator arms 16a to 16d and the stand 14 are not sterile. The portions of the manipulator 12 projecting into the sterile surgical area 40, i.e. the manipulator arms 16a to 16d with coupling units, the stand head 20, and a portion of the stand arm 28, are wrapped in a sterile manner in a sterile barrier 42 indicated by the dash line, such as a sterile flexible wrap or a sterile foil, so that they can be safely arranged in the sterile surgical area 40. The input and output unit 38 is located outside the sterile area 40 and therefore does not require sterile packaging.

In a large number of operations, the instruments units 100a to 100d must be changed several times during the operation due to the course of the operation. Thus, a sterile interface must be provided between the manipulator arm 16a to 16d and the instrument unit 100a to 100d to ensure that the non-sterile coupling unit of the manipulator arm 16a to 16d is covered in a sterile manner even after the instrument unit 100a to 100d is disconnected.

In addition, the sterile instrument unit 100a to 100d must not come into direct contact with non-sterile parts of the coupling unit or the manipulator arm 16a to 16d to prevent contamination of the sterile instrument unit 100a to 100d and the sterile area 40 before and/or after the instrument unit 100a to 100d is separated from the manipulator arm 16a to 16d. This allows the instrument unit 100a to 100d to be deposited in the sterile area 40 without contaminating other elements in the sterile area 40. The sterile barrier 42 is configured to package and hermetically separate and shield the manipulator arm 16a to 16d, the coupling unit, the stand head 20, and at least portions of the stand arm 28 from the sterile field. For this purpose, the sterile barrier 42 may be assembled from a plurality of individual elements, with seams between the individual elements fabricated, for example welded or bonded, to be impermeable and allow sterile separation of the sterile area from non-sterile units. Alternatively, the sterile barrier 42 is seamlessly fabricated from a single piece.

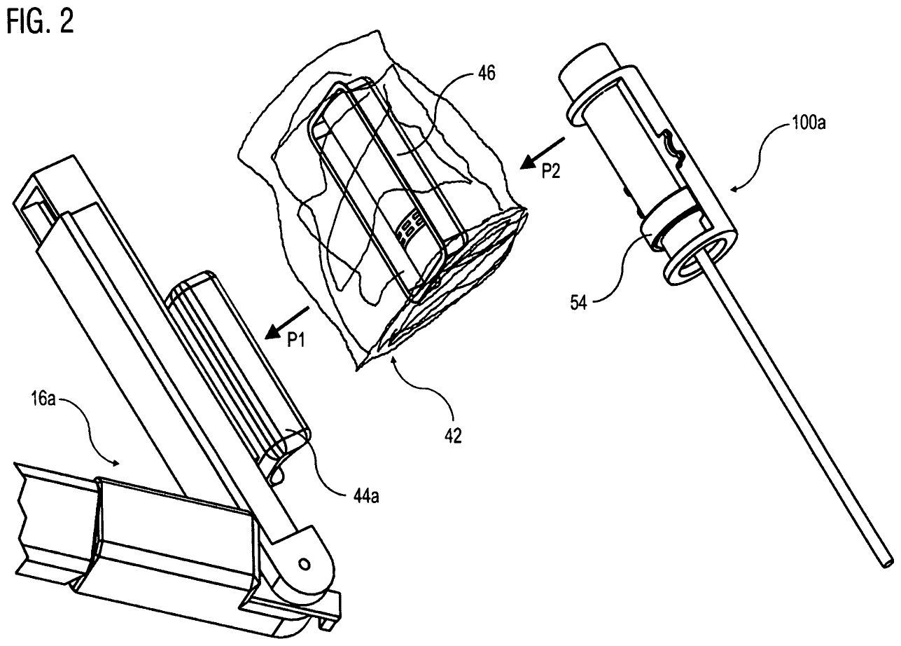

FIG. 2 shows a portion of the manipulator arm 16a of the device of FIG. 1 with a coupling unit 44a, and the sterile barrier 42 and instrument unit 100a in an uncoupled state. Elements having the same structure and/or function have the same reference signs in further Figures. In the following, only the manipulator arm 16a with the instrument unit 100a and the coupling unit 44a are described. However, the explanations also apply to the manipulator arms 16b to 16d, which have substantially the same structure, and the instrument units 100b to 100d connected thereto.

The coupling unit 44a comprises a coupling unit drive element not visible in FIG. 2. The instrument unit 100a includes a corresponding instrument drive element 54. The coupling unit drive element may also be generally referred to as the first drive element and the instrument drive element 54 may also be generally referred to as the second drive element.

In FIG. 2, the sterile barrier 42 is in a collapsed state. In the embodiment according to FIG. 2, the sterile barrier 42 comprises a flexible part, for example a sterile foil, and a dimensionally stable part 46. The dimensionally stable part 46 of the sterile barrier 42 is manufactured to fit precisely and is elastically deformable at least in certain areas to such an extent that it can be pulled over the coupling unit 44a and, in a joined state, bears tightly against at least part of an enveloping surface of the coupling unit 44a. In particular, one side of the part 46 bears tightly against the surface of the coupling unit 44a facing the instrument unit 100a. The flexible part of the sterile barrier 42 is put over the manipulator arm 16a.

The flexible part of the sterile barrier 42 and the dimensionally stable part 46 are joined together to form a continuous sterile barrier between the sterile area 40 and the manipulator arm 16a, the coupling unit 44a and the stand arm 28.

Before coupling the instrument unit 100a to the coupling unit 44a, the sterile barrier 42 is pulled over the manipulator arm 16a and the dimensionally stable part 46 is pulled over the coupling unit 44a in the direction of the arrow P1. For coupling, the instrument unit 100a is then connected to the coupling unit 44a in the direction of the arrow P2.

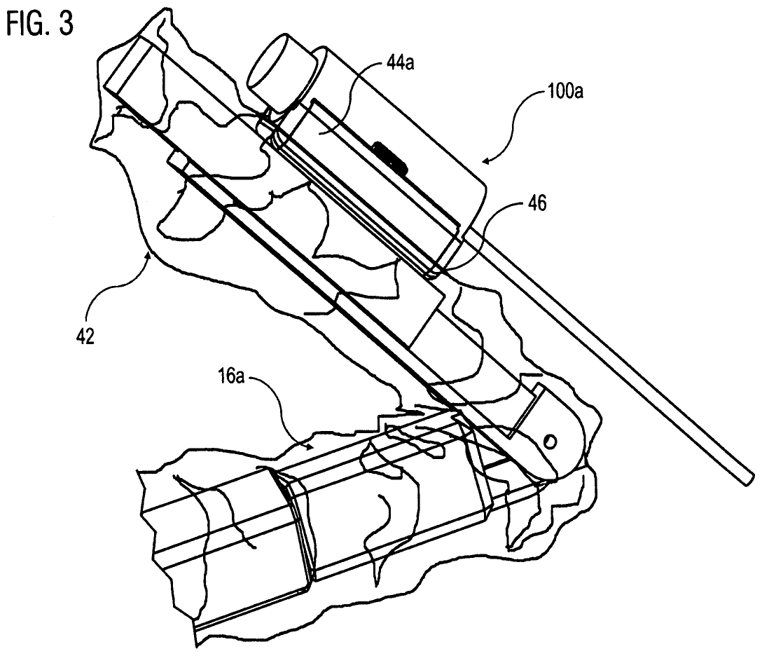

FIG. 3 shows the part of the manipulator arm 16a according to FIG. 2 with the coupling unit 44a as well as the sterile barrier 42 and the instrument unit 100a in a coupled state. In the coupled state, the sterile barrier 42, in particular the dimensionally stable part 46, is pulled over the coupling unit 44a and the manipulator arm 16a. Subsequently, the instrument unit 100a has been guided to a connecting region of the coupling unit 44a so that the connecting region of the coupling unit 44a and a connecting region of the instrument unit 100, separated from the sterile barrier 42, are arranged opposite to each other. The instrument unit 100a can be reversibly attached to the coupling unit 44a with the aid of tabs. Alternatively or additionally, the instrument unit 100a may be attached to the coupling unit 44a with the aid of magnets or another suitable attachment.

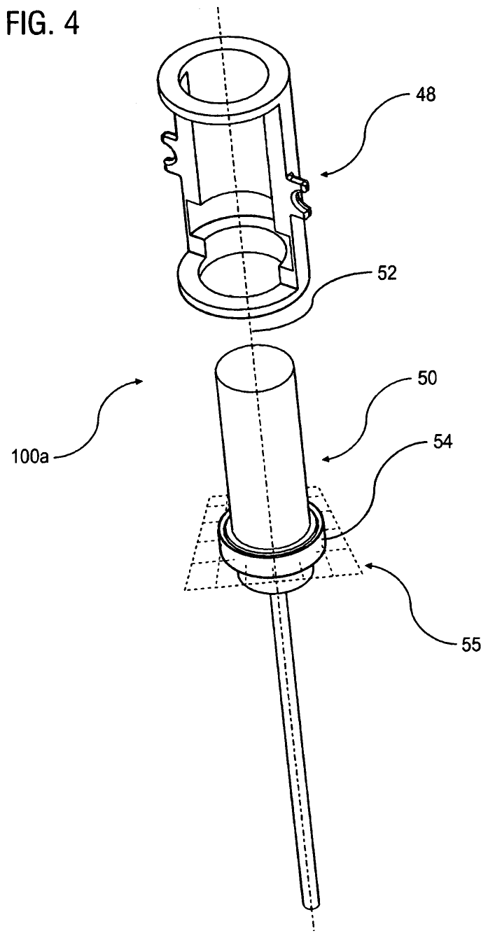

FIG. 4 shows a schematic exploded view of the instrument unit 100a with an outer part 48 and an inner part 50. In an assembled state of the instrument unit 100a, the inner part 50 is rotatably mounted within the outer part 48. The inner part 50 is rotatable about an axis of rotation 52 relative to the outer part 48, in particular, the instrument drive element 54 is rotatable about the axis of rotation 52 relative to the outer part 48. Furthermore, the instrument drive element 54 is arranged in a plane of rotation 55 and rotates in this plane 55. The plane 55 is orthogonal to the axis of rotation 52. In the coupled state, the outer part 48 is stationarily connected to the coupling unit 44a, whereas the inner part 50 is freely rotatable relative to the outer part 48.

Alternatively, the instrument unit 100a may comprise only the inner part 50, and the inner part 50 may be rotatably mounted in the coupling unit 44a. In a further alternative, the instrument unit 100a may comprise only the inner part 50, wherein the inner part 50 is connected to the coupling unit 44a in a rotationally fixed manner and the instrument drive element 54 of the instrument unit 100a is rotatably mounted within the inner part 50.

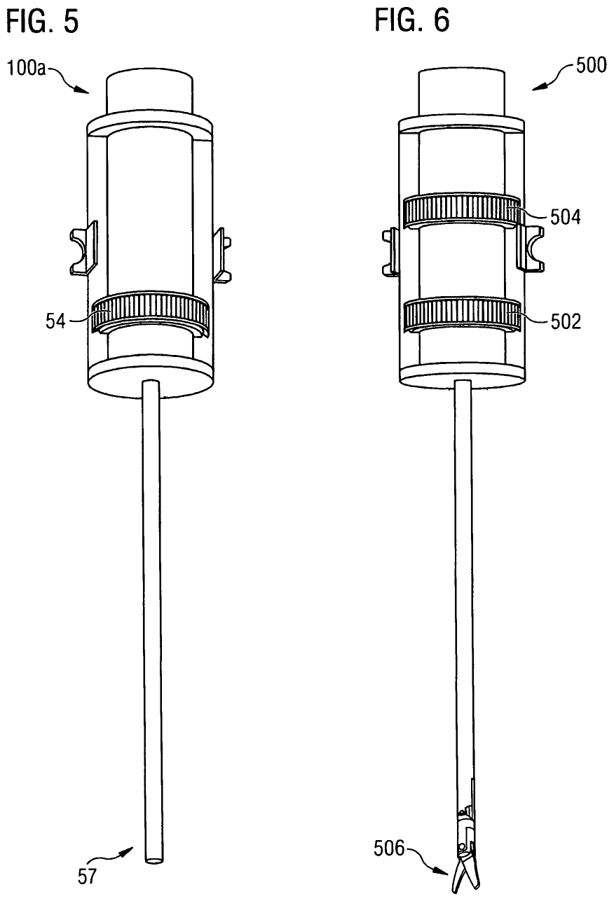

FIG. 5 shows a side view of the instrument unit 100a with the instrument drive element 54 in an assembled state. With the aid of the instrument drive element 54, an end effector may be moved and/or controlled with one degree of freedom. The instrument drive element 54 is mechanically connected to an end effector so that rotation of the instrument drive element 54 can move and/or control the end effector. The end effector may be connected to the instrument drive element 54 with the aid of a gear. For example, a rotational movement of the instrument drive element 54 can thus be converted into a linear movement along the axis of rotation 52. Preferably, the end effector is arranged at an end 57 of a shaft of the instrument unit 100a. In the embodiment, the end effector is an optical system of an endoscope and has one degree of freedom. With the aid of the instrument drive element 54, the optical system of the endoscope and/or the image acquisition sensor(s) of the endoscope can be rotated or pivoted, for example. Preferably, the entire endoscope is rotated for this purpose.

Alternatively, the end effector may be a surgical instrument, such as scissors, a needle holder, a clamp, or forceps.

FIG. 6 shows an instrument unit 500 according to a second embodiment with a first instrument drive element 502 and a second instrument drive element 504. In this case, an end effector may be movable and/or controllable in two degrees of freedom. The end effector of the instrument unit 500 is a pair of scissors 506 that can be opened and closed, for example. In FIG. 6, the pair of scissors 506 is shown in an open state.

Alternatively, the end effector may be, for example, an optical system of an endoscope, which is both rotatable about the axis of rotation 52 and movable with the aid of a joint about an axis orthogonal to the axis of rotation 52.

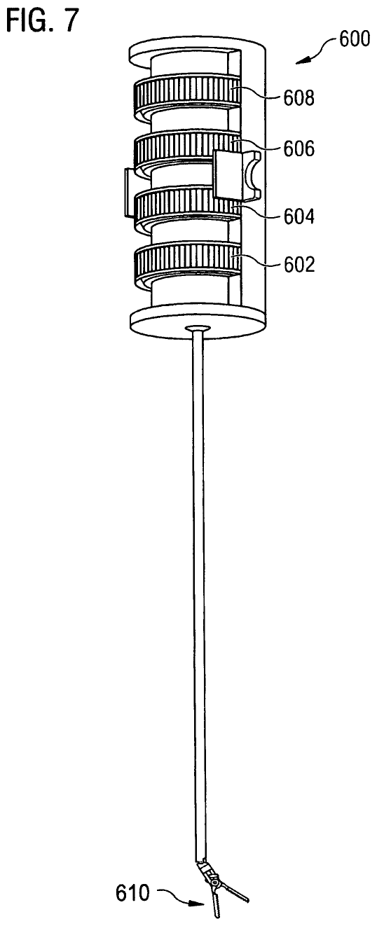

FIG. 7 shows an instrument unit 600 according to a third embodiment with altogether four instrument drive elements 602, 604, 606, 608. In this case, an end effector may be movable and/or controllable in four degrees of freedom. For example, the end effector may be a pair of scissors or a pair of forceps. The end effector of the instrument unit 600 is a pair of forceps 610. In this case, for example, both the levers or legs of the pair of forceps 610 are movable relative to each other and the pair of forceps 610 is rotatable and/or tiltable about an X-axis, Y-axis and/or Z-axis. This allows easy and precise positioning of the end effector relative to the operation field. In FIG. 7, the pair of forceps 610 is shown in an open state, as well as tilted out of the axis 52.

The axis of rotation 52 runs through the circle centers of the four instrument drive elements 602, 604, 606, 608 of the instrument unit 600. The instrument drive elements 602, 604, 606, 608 are arranged in parallel planes of rotation that are orthogonal to the axis of rotation 52.

Each of the instrument units 100a, 500, 600 may be associated with an identification element that can be read by a readout unit in the coupling unit 44a. For example, an instrument unit 100a, 500, 600 may comprise an RFID element that can be read out in a non-contact manner using the coupling unit 44a. The identification element may include information about the instrument unit 100a, 500, 600, such as number of degrees of freedom and number of instrument drive elements, range of motion of each instrument drive element, and type of end effector. In addition, information about the state of the instrument unit 100a, 500, 600 may be communicated, in particular whether the instrument unit 100a, 500, 600 is unused and sterile, to prevent use of contaminated instrument units 100a, 500, 600.

In addition, further interfaces may be provided between the coupling unit 44a and the instrument unit 100a, 500, 600 for information transfer. Preferably, the interfaces are non-contact interfaces, for example optical interfaces and/or interfaces based on other electromagnetic waves.

In other embodiments, also other end effectors may be used and driven in the instrument units 500 and 600.

Alternative modes of driving are described below for driving an instrument drive element of an instrument unit with the aid of a coupling unit.

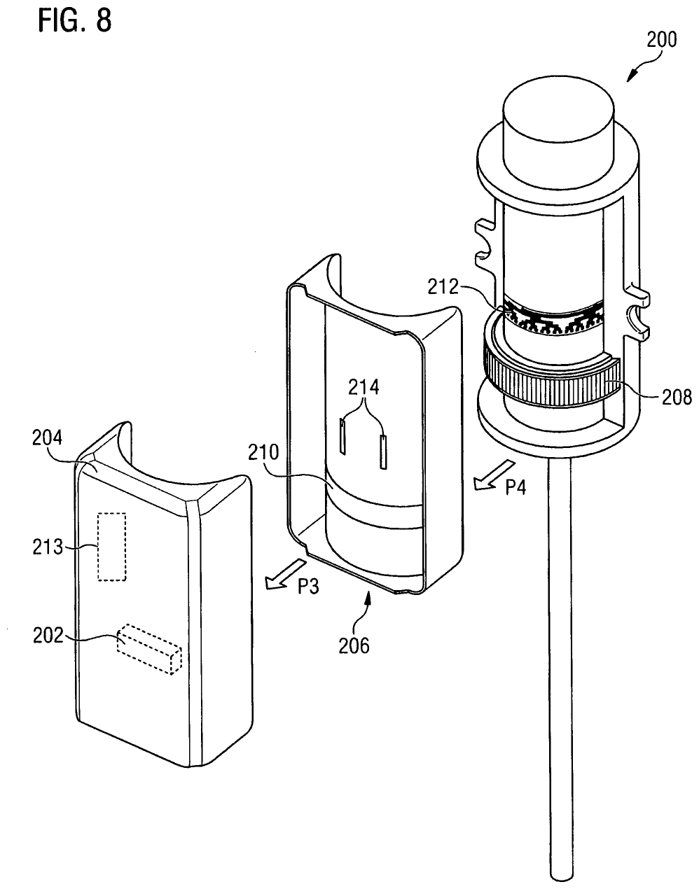

FIGS. 8 to 10 respectively show an instrument unit 200 and a coupling unit 204 according to a fourth embodiment with an electromagnetic drive. FIG. 8 shows a coupling unit drive element 202 in the coupling unit 204. A part 206 of the sterile barrier 42 is put over the coupling unit 204 in the direction of the arrow P3. Subsequently, the instrument unit 200 is coupled to the coupling unit 204 in the direction of the arrow P4.

The instrument unit 200 comprises an instrument drive element 208. In the coupled state, the coupling unit drive element 202 and the instrument drive element 208 are arranged side by side in the plane of rotation 55 orthogonally to the axis of rotation 52. The dimensionally stable part 206 of the sterile barrier 42 is configured and arranged such that in a region 210 along the plane of rotation 55 of the drive elements 202, 208, the dimensionally stable part 206 of the sterile barrier 42 bears tightly against the coupling unit drive element 202 and does not come into contact with the instrument drive element 208. In particular, the dimensionally stable part 206, the coupling unit drive element 202 and the instrument drive element 208 are each configured and arranged relative to each other such that the distance between the coupling unit drive element 202 and the instrument drive element 208 in the coupled state is 0.05 mm to 2 mm and the dimensionally stable part 206 does not contact the rotating instrument drive element 208.

Additionally, the instrument unit 200 includes an optically detectable pattern 212 that is associated with the instrument drive units 208 and rotates with the instrument drive element 208 about the axis of rotation 52 upon rotation of the instrument drive element 208. The pattern 212 is detected in the coupled state of the coupling unit 204 and the instrument unit 200 with the aid of one or more optical sensors of the coupling unit drive element 202, wherein a detectable angle of rotation of the instrument drive element 208 about the axis of rotation 52 is determined by a reading unit 213 in a non-contact manner. The detectable angle of rotation is determined by a resolution of the optical sensor and a resolution of the pattern. The pattern 212 is formed and arranged around the circumference of the instrument drive element 208 such that the optical sensor detects a uniquely distinguishable part of the pattern 212 for each detectable angle of rotation. This part of the pattern 212 is different from other parts of the pattern 212 that can be detected at further angles of rotation. In particular, the pattern 212 can be a so-called Gray code. Thus, an unambiguous position determination of the instrument drive element 208 about the axis of rotation 52 is possible. In order to ensure reliable optical detection of the pattern 212, at least individual areas 214 made of optically transparent material are provided in the part 206.

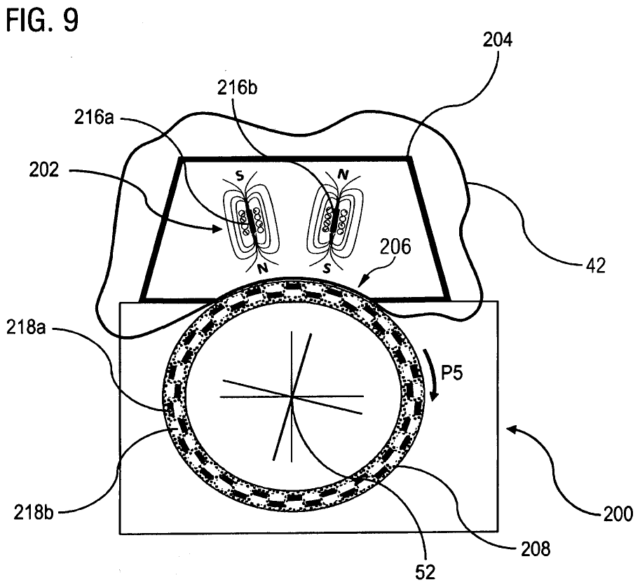

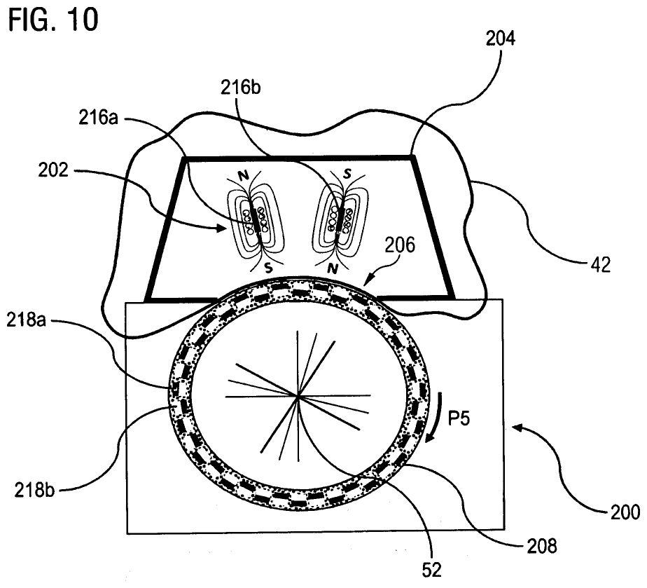

The coupling unit drive element 202 and the instrument drive element 208 comprise magnetic and/or magnetic field-generating elements in the embodiment according to FIG. 8. FIGS. 9 and 10 show schematic sectional views of the electromagnetic drive principle according to FIG. 8, wherein the sectional plane is the plane of rotation 55. The coupling unit 204 includes several magnetic field-generating elements 216a, 216b as part of the coupling unit drive element 202. These elements 216a, 216b are, for example, electromagnets. In the schematic drawings in FIGS. 9 and 10, two magnetic field-generating elements 216a, 216b are shown in each case, alternatively a larger number of elements 216a, 216b is possible. The elements 216a, 216b are arranged in the plane of rotation 55 around a part of the circumference of the instrument drive element 208. The coupling unit drive element 202 surrounds the instrument drive element 208 at an angle of 45° to 180° about the axis of rotation. The polarity of the magnetic and magnetic field-generating elements is exemplarily identified with N (north pole) or S (south pole) in FIGS. 8 to 10.

The instrument drive element 208 includes a plurality of permanent magnets 218a, 218b along the circumference of the drive element 208. These magnets 218a, 218b have alternating opposite polarizations. The movement of the instrument drive element 208 is generated by the coupling unit drive element 202 according to the principle of an electric motor, in particular a stepper motor. Here, the coupling unit drive element 202 is the stator and the instrument drive element 208 is the rotor. The magnetic field-generating elements 216a, 216b are arranged in the coupling unit drive element 202 is a non-movable, i.e. stationary, manner. In the embodiment according to FIGS. 8 to 10, the instrument drive element 208 has a cylindrical shape. Alternatively, the lateral surface may merely be substantially cylindrical and may, for example, be a shape having a base of a regular polygon centered on the axis of rotation 52. At least an enveloping surface enclosing this polygon is then cylindrical.

With the aid of the magnetic field-generating elements 216a, 216b, the coupling unit drive element 202 generates an electromagnetic field. The elements 216a, 216b are controlled such that they change their state with each step. FIG. 9 shows a first state with a first polarization of the magnetic field-generating elements 216a, 216b. FIG. 10 shows a second state with a second, respectively reversed polarization of the magnetic field-generating elements 216a, 216b. As a result, the coupling unit drive element 202 generates a stepwise moving electromagnetic field. The generated electromagnetic field acts with a force on the magnetic elements 218a, 218b of the instrument drive element 208. Thus, each change in polarization of the elements 216a, 216b rotates the instrument drive element 208 by an angle in the direction of the arrow P5 about the axis of rotation 52 due to the magnetic force. The rotation of the instrument drive element 208 can be translated into a desired movement and/or control of the end effector with the aid of gears.

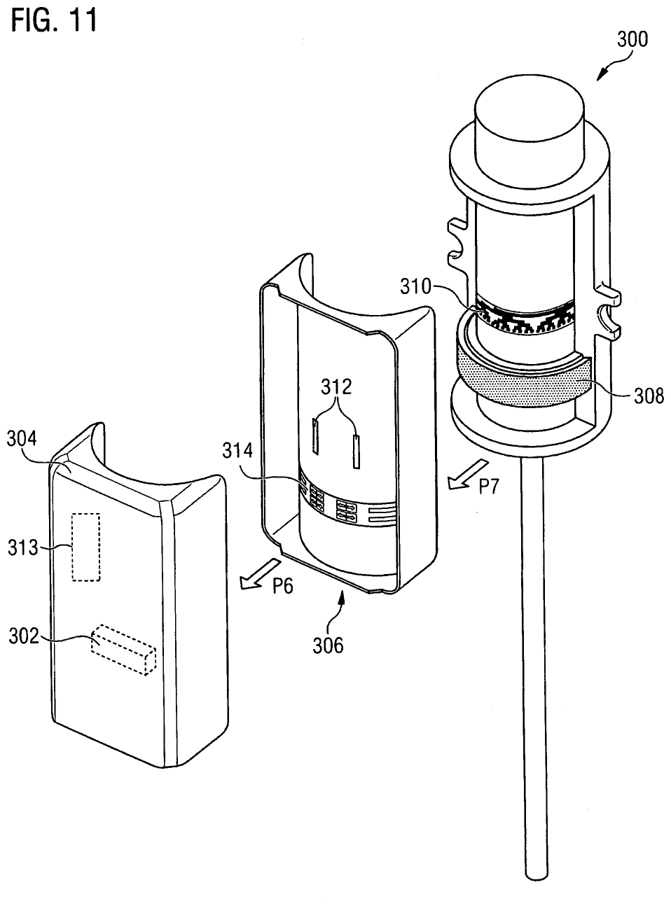

FIGS. 11 to 13 show an instrument unit 300 and a coupling unit 304 according to a fifth embodiment having a drive with a mechanical power transmission. The mechanical power transmission is achieved in particular with the aid of a frictional and/or positive connection. The drive may, for example, be designed as a piezo motor. Alternatively, the mechanical power transmission can be achieved with the aid of camshafts or coupling gears.

FIG. 11 shows a coupling unit drive element 302 in the coupling unit 304. A dimensionally stable part 306 of the sterile barrier 42 is put over the coupling unit 304 in the direction of the arrow P6. The instrument unit 300 may then be coupled to the coupling unit 304 in the direction of arrow P7. The instrument unit 300 comprises an instrument drive element 308. In the coupled state, the coupling unit drive element 302 and the instrument drive element 308 are arranged side by side along the plane of rotation 55 orthogonally to the axis of rotation 52.

The instrument unit 300 includes an optically detectable pattern 310. This pattern 310 is detected in the coupled state with the aid of one or more optical sensors of the coupling unit drive element 302 and thus a detectable angle of rotation of the instrument drive element 308 about the axis of rotation 52 is determined by a reading unit 313 in a non-contact manner. As described further above for instrument unit 200, the pattern 310 is formed and arranged around the circumference of the instrument drive element 308 such that the optical sensor detects a uniquely distinguishable part of the pattern 310 for each detectable angle of rotation. The pattern 310 can in particular be a so-called Gray code. Thus, an unambiguous position determination of the instrument drive element 308 about the axis of rotation 52 is possible. To ensure reliable optical detection of the pattern 310, areas 312 made of optically transparent material are provided in the dimensionally stable part 306.

In the embodiment according to FIG. 11, the coupling unit drive element 302 has movable engagement elements which, in the coupled state of the instrument unit 300 and the coupling unit 304, are at least temporarily in engagement with the lateral surface of the instrument drive element 308. To ensure the integrity of the sterile barrier 42 between the drive elements 302, 308, in particular the dimensionally stable part 306, a flexible region 314 is provided in the dimensionally stable part 306 which is configured to allow movement and/or deformation of the engagement elements without being damaged.

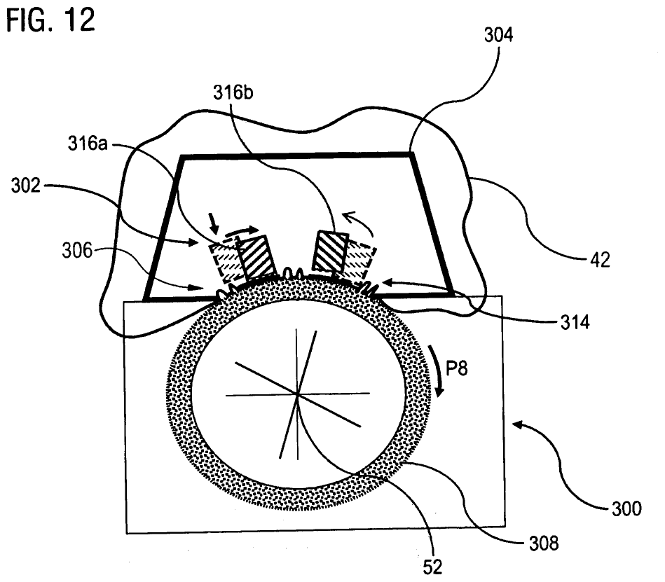

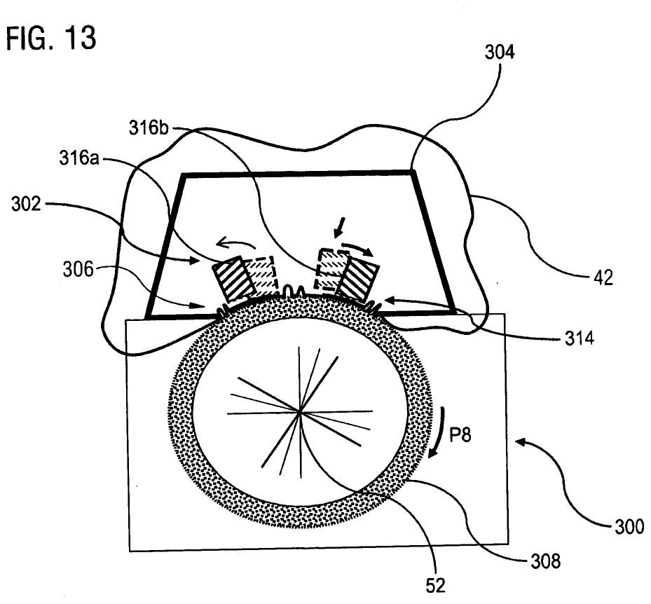

FIGS. 12 and 13 show schematic sectional views of the mechanical drive principle according to FIG. 11, wherein the sectional plane is the axis of rotation 55. The coupling unit 304 comprises the coupling unit drive element 302 with several engagement elements 316a, 316b. These elements 316a, 316b are movable and/or deformable and at least temporarily in engagement with the lateral surface of the instrument drive element 308. In the schematic drawings according to FIGS. 12 and 13, two engagement elements 316a, 316b are illustrated, alternatively a higher number of engagement elements 316a, 316b is possible. The engagement elements 316a, 316b are arranged in the plane of rotation 55 around a portion of the circumference of the instrument drive element 308. Further, the engagement elements 316a, 316b are movable and/or deformable in the plane of rotation 55.

The surface of the lateral surface of the instrument drive element 308 is configured to allow the engagement elements 316a, 316b to form a frictional or positive connection with the lateral surface. In the embodiment according to FIGS. 11 to 13, the instrument drive element 308 has a cylindrical shape, with the lateral surface oriented parallel to the side of the coupling unit drive element 302 that faces the instrument drive element 308 in the coupled state. Alternatively, the lateral surface may merely be substantially cylindrical and/or oriented substantially parallel to the coupling unit drive element 302. For example, the surface of the lateral surface of the instrument drive element 308 is roughened or has a toothed rim to enable a secure force and/or positive connection with the engagement element.

In order to rotate the instrument drive element 308 about the axis of rotation 52 using the coupling unit drive element 302, an engagement element 316a, 316b is in positive or frictional connection with the instrument drive element 308, respectively. By moving the engagement element 316a, 316b about the axis of rotation 52, the instrument drive element 308 rotates about the axis of rotation 52 in the direction of the arrow P8. The rotation of the instrument drive element 308 can be translated into movement and/or control of the end effector using gears. In FIG. 12, in a first step, the engagement element 316a moves in the direction of the instrument drive element 308, enters into a frictional connection with the instrument drive element 308, and subsequently rotates the instrument drive element 308 in the direction of the arrow 8. At the same time, the engagement element 316b disengages from the instrument drive element 308 and moves back to an initial position opposite to the direction of rotation P8. In FIG. 13, in a second step, the engagement element 316b moves in the direction of the instrument drive element 308, enters into a frictional connection with the instrument drive element 308, and rotates the instrument drive element 308 in the direction of the arrow P8. Analogously to FIG. 12, meanwhile the engagement element 316a disengages from the instrument drive element 308 and moves back to an initial position against the direction of rotation P8. Preferably, at least one engagement element 316a, 316b is always in frictional and/or positive connection with the instrument drive element 308 so that the instrument drive element 308 is secured against unintentional rotation.

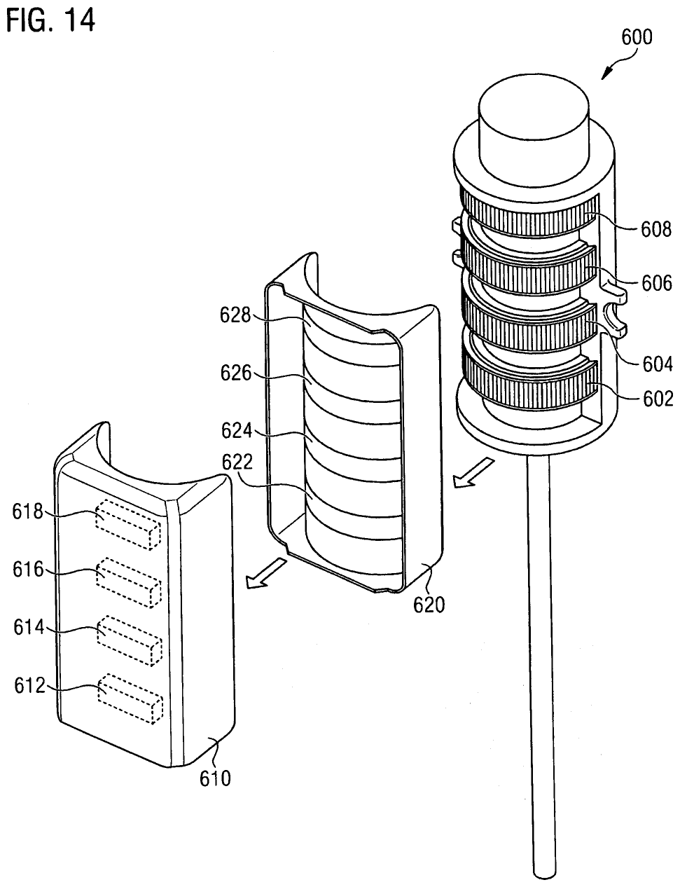

FIG. 14 shows the instrument unit 600 according to the embodiment of FIG. 7 and a coupling unit 610. The instrument unit 600 comprises four instrument drive elements 602, 604, 606, 608. The coupling unit 610 comprises four coupling unit drive elements 612, 614, 616, 618. The coupling unit drive elements 612, 614, 616, 618 are arranged one above the other in a plane of rotation of an instrument drive element 602, 604, 606, 608. Thus, the coupling unit drive elements 612, 614, 616, 618 are arranged in a line parallel to the axis of rotation 52 of the instrument unit 600. Further, each coupling unit drive element 612, 614, 616, 618 is arranged opposite along at least a portion of the enveloping surface of an instrument drive element 602, 604, 606, 608.

Each instrument drive element 602, 604, 606, 608 may have a circumferential pattern associated therewith for sensing the angle of rotation of the instrument drive element 602, 604, 606, 608 about the axis of rotation 52, according to the method described further above. Further, a dimensionally stable part 620 of a sterile barrier may comprise an optically transparent region to allow the pattern to be detected using an optical sensor.

The dimensionally stable part 620 of the sterile barrier further comprises several regions 622, 624, 626, 628, that are configured to either allow movement and/or deformation by engagement elements without being damaged or to bear tightly against the coupling unit drive elements 612, 614, 616, 618 and not come into contact with the instrument drive elements 602, 604, 606, 608, depending on the type of drive. In the case of a drive according to the fifth embodiment in FIGS. 11 to 13, at least intermittent contact of the regions 622, 624, 626, 628 is provided when an engagement element is in frictional connection and/or positive connection with an instrument drive element 602, 604, 606, 608.

The number of coupling unit drive elements 612, 614, 616, 618 and instrument drive elements 602, 604, 606, 608 may differ in other embodiments, as described further above in the previous embodiments. At least one coupling unit drive element and one instrument drive element are provided in each case. Furthermore, the coupling unit comprises at least as many coupling unit drive elements as the number of instrument drive elements of the instrument unit. Thus, in the coupled state, each coupling unit drive element is paired with an instrument drive element in a plane of rotation 55.

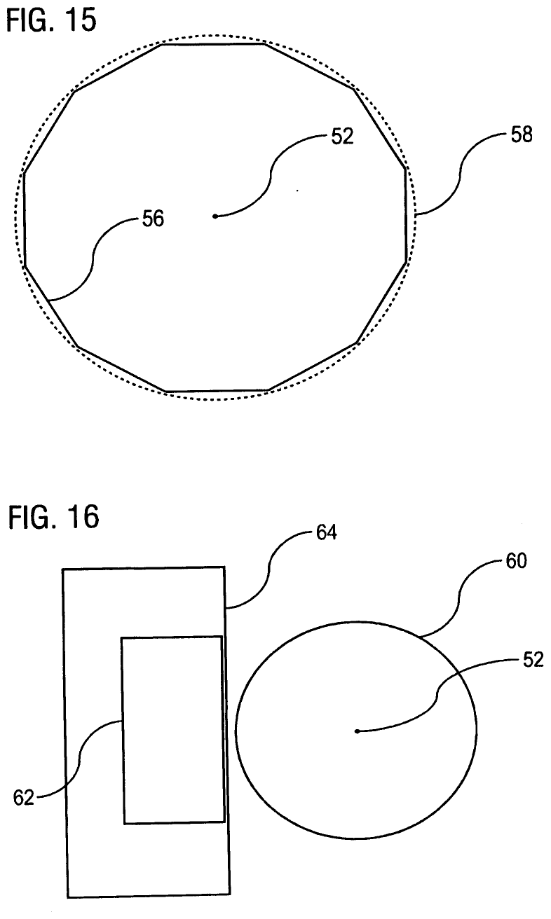

FIG. 15 shows a schematic sectional drawing of an instrument drive element 56 according to a sixth embodiment of an instrument unit, wherein the sectional plane is the plane of rotation 55. The instrument drive element 56 has a shape with a base of a regular dodecagon polygon and a center on the axis of rotation 52. An assumed enveloping surface 58 has a cylindrical shape with a center on the rotation axis 52 and a radius selected such that the enveloping surface 58 precisely encloses the polygon.

FIG. 16 shows a generalized schematic representation of the drive of an instrument unit as a sectional drawing along the plane of rotation 55. An instrument drive element 60 is rotated with the aid of a coupling unit drive element 62 about the axis of rotation 52. A sterile barrier 64 surrounds the coupling unit drive element 62. In particular, the sterile barrier 64 is arranged continuously between the coupling unit drive element 62 and the instrument drive element 60.

What is claimed is:

1. A device for robot-assisted surgery,
with at least one manipulator arm with a non-sterile coupling unit comprising at least one first drive element,
with a sterile instrument unit arranged in a sterile area and comprising at least one second drive element arranged rotatably around an axis of rotation,
wherein the instrument unit is couplable with the coupling unit of the manipulator arm,
wherein the first drive element and the second drive element are configured and arranged in a coupled state such that by the first drive element a force can be exerted on the second drive element, for rotation of the second drive element about the axis of rotation,
wherein in the coupled state the first drive element and the second drive element are arranged side by side in a plane of rotation orthogonally to the axis of rotation, and
with a sterile barrier which is arranged at least between the first drive element and the second drive element.

2. The device according to claim 1, characterized in that in the coupled state, the first drive element is arranged in the plane of rotation of the second drive element such that a surface of the first drive element facing the second drive element runs parallel to a cylindrical lateral surface or a cylindrical enveloping surface of the second drive element in at least one area.

3. The device according to claim 1, characterized in that in the plane of rotation at a side of the first drive element facing the second drive element magnetic field-generating elements are arranged and magnetic field-generating or magnetic elements of the second drive element are arranged on a circular path around the axis of rotation.

4. The device according to claim 3, characterized in that the magnetic field-generating elements of the first drive element are electromagnets and the magnetic field-generating elements of the second drive element are electromagnets with circumferentially alternating poles, or that the magnetic field-generating elements of the first drive element are electromagnets and the magnetic elements of the second drive element are permanent magnets with circumferentially alternating poles.

5. The device according to claim 4, characterized in that magnetic fields can be generated in the first drive element with the aid of the electromagnets, by which magnetic fields the force can be exerted on the permanent magnets or the electromagnets of the second drive element, wherein the second drive element rotates by changing the polarity of the electromagnets and/or changing a switching state of the electromagnets of the first drive element.

6. The device according to claim 1, characterized in that the first drive element comprises at least one movable and/or deformable actuator, which is movable and/or deformable such that the actuator contacts a lateral surface of the second drive element at least temporarily such that the force can be exerted on the second drive element by frictional connection and/or positive connection between the actuator and the lateral surface of the second drive element.

7. The device according to claim 6, characterized in that the actuator is at least one piezoelectric actuator and with which, by way of a frictional connection and/or positive connection between the actuator and the lateral surface of the second drive element, the force can be exerted on the second drive element.

8. The device according to claim 1, characterized in that the surface of the first drive element facing the second drive element runs along an arc about the axis of rotation with a center angle of 45° to 180°.

9. The device according to claim 1, characterized in that the sterile barrier has a continuously closed surface at least in the area between the first drive element and the second drive element.

10. The device according to claim 1, characterized in that a section of the sterile barrier arranged between the first drive element and the second drive element runs along an arc around the axis of rotation.

11. The device according to claim 1, characterized in that the coupling unit comprises at least one sensor unit for detecting an angle of rotation of the second drive element.

12. The device according to claim 11, characterized in that the sensor unit comprises an optical sensor which detects a coded, optically detectable pattern on the second drive element circulating in a plane of rotation, each detectable angle of rotation of the second drive element being assigned a part of the pattern which is detectable by the sensor at this angle of rotation, each part of the pattern which is detectable by the sensor at one angle of rotation being uniquely different from other parts of the pattern which are detectable by the sensor at other angles of rotation.

13. The device according to claim 1, characterized in that in a coupled state, the second drive elements are arranged in a plurality of planes of rotation one above the other along the same axis of rotation, and each first drive element is arranged in a plane of rotation in pairs with a second drive element, the number of pairs of drive elements being at least two, in particular three or four.

14. The device according to claim 13, characterized in that a plurality of sensor units are provided, that the number of sensor units corresponds at least to the number of second drive elements, and in that at least one sensor unit in each case detects the angle of rotation of a second drive element.

15. The device according to claim 1, characterized in that the instrument unit comprises an instrument with an end effector arranged at a distal end of an instrument shaft, wherein the at least one second drive element is coupled to the end effector and wherein the end effector is movable and/or controllable in at least one degree of freedom with the aid of the at least one second drive element, in particular, in the case of four second drive elements, in four degrees of freedom, wherein in each case two of the second drive elements effect a rotary movement about the longitudinal axis of the instrument shaft and in each case two further second drive elements effect a longitudinal movement in the direction of the longitudinal axis of the instrument shaft or that the instrument unit comprises an endoscope with an endoscope shaft, wherein the at least one second drive element is coupled to the endoscope, the endoscope shaft and/or an optical system of the endoscope such that a movement of the endoscope, the endoscope shaft and/or the optical system is possible in at least one degree of freedom with the aid of the at least one second drive element.
